(19) United States
(12) Patent Application Publication
(10) Pub. No.: US 2011/0281811 A1
(43) Pub. Date: Nov. 17, 2011

(54) Use Of Leukotriene Inhibitors For Treating Lung Diseases In Prematurely Born Infants

(76) Inventor: Sabine Rupprecht, Uttenreuth (DE)

(21) Appl. No.: 13/129,044

(22) PCT Filed: Nov. 12, 2009
(86) PCT No.: PCT/EP09/65026

§ 371 Date: Aug. 1, 2011

(30) Foreign Application Priority Data
Nov. 12, 2008 (DE) 10 2008 056 875.9

(51) Int. Cl. A61K 31/47; A61K 31/41; C07D 215/14; C07D 209/18; C07D 257/04; A61K 31/7048; A61K 31/522; A61K 31/5415; A61K 31/585; A61K 31/439; A61K 31/515; A61K 31/4166; A61K 31/496; A61P 11/00; A61P 11/08; A61P 29/00; A61P 31/00; A61P 11/06; A61P 25/00; A61P 7/10; A61P 9/08; A61K 31/4045

(52) U.S. Cl. 514/29; 514/311; 514/415; 514/382; 546/174; 548/491; 548/253; 514/263.32; 514/224.2; 514/171; 514/299; 514/271; 514/391; 514/254.11

(57) ABSTRACT

The invention relates to the use of at least one leukotriene inhibitor for preparing a pharmaceutical composition for the prophylaxis and/or treatment of lung diseases, more particularly of bronchopulmonary dysplasia, in prematurely born infants. The invention further relates to a pharmaceutical composition for the prophylaxis and/or treatment of lung diseases, more particularly of bronchopulmonary dysplasia, in prematurely born infants, the pharmaceutical composition comprising at least one leukotriene inhibitor. The invention additionally relates to a method for the treatment and/or prophylaxis of a lung disease, more particularly of a bronchopulmonary dysplasia, in a prematurely born infant, where the prematurely born infant is administered a pharmacologically active amount of at least one leukotriene inhibitor.

The invention relates to possibilities of treatment of lung diseases in prematurely born infants.

Due to the technical progresses of the medicine in the last decades, there are more and more prematurely born infants born alive, which are subject to the risk of development of a lung disease not least because of their prematurity. In particular, the so-called bronchopulmonary dysplasia (BPD) occurs in prematurely born infants comparatively often since these infants usually have to be given artificial respiration over a longer period of time, for example to treat the newborn respiratory distress syndrome. The incidence of the BPD continues to remain high due to this fact and even further increases. In Germany, for example approximately 1300 to 1500 newborns annually come down with BPD, wherein the incidence thereof is at 1 to 1.8 cases per 1000 infants born alive and presents one of the most frequent chronic lung diseases of the infanthood. The BPD is associated with a series of complications and is accompanied by an increased rate of mortality. The most frequent acute complications are:

1. Oxygen and ventilation dependency for weeks up to months by reduced oxygenation function of the lung;

2. Cumulative occurrence of pulmonary and systemic infections probably caused by mucus retention in badly aerated lung sections;

3. Pneumothorax and lung emphysema by overstretching individual areas under ventilation; and

4. Pulmonary hypertension by vascular constriction in the badly aerated lung sections, whereby cor pulmonale with resulting heart failure may arise as a result.

Dystrophy, psychomotor developmental disorders as well as rehospitalizations due to recurrent pulmonary infections especially in the first year of life belong to the long-term consequences. It is particularly problematic that the disease itself progresses in many infants even if the causing factors have been eliminated, and finally may result in complete destruction of the lung with lethal consequence.

The previous concept of therapy of the BPD considers the already known pathophysiological causes of this disease and relies on various therapeutic basic principles. They involve the prenatal prevention with glucocorticoides for supporting the lung maturation and then extend in case of manifest BPD from oxygen application via liquid restriction, optionally associated with employment of diuretics. Furthermore, inhalation therapies as well as new systemic administration of glucocorticoides are provided as a possible treatment regimen. Therein, the object of the treatments is always the avoidance of a barotrauma of the lung by ventilation as gentle as possible and early resorting to a so-called continuous positive airway pressure ventilation (CPAP ventilation) as well as the generous indication for surfactant substitution.

Despite of this extensive concept of therapy, the mortality of the BPD actually is at ca. 5-10%. The most frequent causes of death in the first year of life relate to cardiopulmonary complications, pulmonary infections, sepsis or the sudden infant death. In the pathogenesis of the BPD, some factors are already considered to be known and investigated, however, some pathogenetic mechanisms are yet further unexplained despite worldwide intensive research work. For example, it is known that perinatal inflammation processes cause increased vascular permeability in the immature lung. For this reason, currently, corticosteroids are employed in high dose due to their anti-inflammatory effect. Further reasons for the development of the BPD are in an initial water retention in the immature lung as well as in the chemical and mechanical stresses by the ventilation with high oxygen contents. In addition, biological damages by microbial pathogens can occur. The alveoli, the airways and the blood vessels of the lung are affected, which constrict and thus may result in increased pressure in the pulmonary circulation and in stress for the right ventricle.

However, it has turned out that severe impairments of the cerebral development of these infants may occur as side effects by the administration of steroids. Besides a severe damage of the lung, usually, high-degree mental retardations occur in up to 50% of the concerned patients.

The object of the present invention is to provide improved possibilities of treatment for prematurely born infants, with the aid of which the progress of disease often crucial to life can be avoided or at least advantageously affected.

According to the invention, the object is solved by use of at least one leukotriene inhibitor for preparing a pharmaceutical composition for prophylaxis and/or treatment of lung diseases in prematurely born infants, by a pharmaceutical composition according to claim 7 for prophylaxis and/or treatment of lung diseases as well as by a method according to claim 13 for treating and/or for prophylaxis of a lung disease in a prematurely born infant. Advantageous configurations with convenient developments of the invention are specified in the respective dependent claims, wherein advantageous configurations of the use are to be considered as advantageous configurations of the pharmaceutical composition or of the method, and vice versa.

According to the invention, a use of at least one leukotriene inhibitor for preparing a pharmaceutical composition for prophylaxis and/or treatment of lung diseases, in particular of bronchopulmonary dysplasia, in prematurely born infants is provided. It has turned out that the progress of disease in lung diseases of prematurely born infants is associated with a severe inflammatory response in the region of the alveolar epithelium. Therein, it has been found that increased intrapulmonary levels of interleukins and other acute phase proteins occur. Leukotrienes are inflammation mediators, which are known in connection with inflammation responses in bronchial asthma. Especially cysteinyl leukotrienes are therein obviously responsible for the development of inflammations, which in turn act constrictive on the airways of the prematurely born infants and weaken their pulmonary function. Unlike the extremely low treatment success in bronchial asthma, surprisingly, it has turned out that leukotriene inhibitors can be employed with great treatment success both for the preventive and for the curative treatment of lung diseases occurring in prematurely born infants, in particular the bronchopulmonary dysplasia, in order to develop an effective anti-inflammatory effect by blocking the leukotriene system and to prevent increased mucoid secretion in the lung. The applicant expects the reason for this surprising effectiveness in the varying causes and the different manifestation forms with respect to bronchial asthma—without being fixed to this opinion. Furthermore, it has turned out that a tissue modification of the lung by fibroblast proliferation and destruction of the extracellular matrix in prematurely born infants are reliably prevented with the aid of leukotriene inhibitors. Therein, the leukotriene inhibitor can basically be used in each pharmacologically effective form. For example, it can be used as a pure substance, as an enantiomer, as diastereomers, as racemic mixture, as pharmacologically acceptable salt, solvated and/or unsolvated. In summary, the use according to the invention thus allows substantially improved possibilities of treatment for prematurely born infants, wherein compared to the previous therapy approaches substantially lower and rarer side effects occur. The progress of disease often crucial to life is therefore avoidable a priori or can at least be considerably alleviated.

In an advantageous development of the invention, it is provided that at least one leukotriene receptor antagonist and/or at least one leukotriene synthesis antagonist is used as the leukotriene inhibitor. Leukotriene receptor antagonists bind to leukotriene receptors (LTD4 receptors) and thereby block the effect of the leukotrienes in the inflammation process. Hereby, an anti-inflammatory effect in the bronchial system of the prematurely born infant is achieved. Alternatively or in addition, a leukotriene synthesis antagonist can also be used as the leukotriene inhibitor, however, the pharmacological effect of which is in the inhibition of the leukotriene synthesis itself. Thus, the development of leukotrienes can advantageously be blocked via various mechanisms.

In a further advantageous development of the invention, it is provided that montelukast and/or zafirlukast and/or pranlukast are used as the leukotriene receptor antagonist and/or zileuton is used as the leukotriene synthesis antagonist. Montelukast, zafirlukast and pranlukast directly intervene in inflammatory processes by binding to the cysteinyl leukotriene receptors present in the airways with high affinity and selectivity and inhibiting the pro-inflammatory effect of leukotrienes by blocking them. Alternatively or additionally, the antineoplastic hydroxyurea derivative zileuton (Zyflo®) can be used as the leukotriene synthesis antagonist, which blocks the synthesis of leukotrienes from arachidonic acid by inhibition of the 5-lipoxygenase. However, it is to be emphasized that basically other than the mentioned leukotriene receptor antagonists or the mentioned leukotriene synthesis antagonist can also be provided.

In order to allow a particularly flexible and customizable prophylaxis or treatment of lung diseases in prematurely born infants, it has proven to be advantageous in further development of the invention if at least one further active agent from the group of the anti-inflammatory agents, antibiotics, antiasthmatics, bronchodilators, betasympathomimetic drugs, diuretics, parasympatholytics, vitamins, cytochrome P450 inductors and/or vaso-dilating agents is used for preparing the pharmaceutical composition. Hereby, in particular, the most frequent acute complications mentioned above under 1. to 4. can be specifically treated. The use of antibiotics for example allows the inhibition of bacterial infections. With the aid of diuretics, the risk of development of pulmonary edema can advantageously be reduced.

Therein, in further development it has proven to be advantageous if clarithromycin, pentoxifylline, vitamin A, provitamin A, hydrochlorothiazide, spironolactone, furosemide, salbutamol, ipratropium bromide, phenobarbital, phenytoin, rifampicin and/or at least one steroid, in particular a corticosteroid and/or a glucocorticoid, is used as a further active agent in preparing the pharmaceutical composition. For example, with the aid of steroids such as dexamethason, a fast improvement of the pulmonary function and reduction of the occurrence of BPD can be achieved, wherein in contrast to the known therapy approaches, due to the synergistic effect with the leukotriene inhibitor substantially lower dosages are necessary to achieve comparable effects. Accordingly, the frequency of possible side effects can be greatly reduced.

Further advantages arise by preparing the pharmaceutical composition for oral and/or intravenous and/or aerogenous and/or rectal administration. Hereby, the pharmaceutical composition can be particularly flexibly administered such that the degree of development of the concerned prematurely born infant can optimally be taken into account.

A further aspect of the invention relates to a pharmaceutical composition for prophylaxis and/or treatment of lung diseases, in particular of bronchopulmonary dysplasia, in prematurely born infants, wherein the pharmaceutical composition includes at least one leukotriene inhibitor. Hereby, improved possibility of treatment for prematurely born infants is allowed, with the aid of which the progress of disease often crucial to life can be avoided or at least advantageously be affected. The advantages of such a pharmaceutical composition with at least one leukotriene inhibitor can be taken from the previous explanations to the use according to the invention. The preferred embodiments and developments presented in connection with the use according to the invention as well as the advantages thereof correspondingly apply to the method according to the invention and vice versa.

By the pharmaceutical composition including a leukotriene receptor antagonist and/or a leukotriene synthesis antagonist as the leukotriene inhibitor, the development of leukotrienes can advantageously be blocked via various mechanisms. Leukotriene receptor antagonists bind to leukotriene receptors (LTD4 receptors) and thereby block the effect of the leukotrienes in the inflammation process. Hereby, an anti-inflammatory effect in the bronchial system of the prematurely born infant is achieved. Alternatively or additionally, a leukotriene synthesis antagonist can also be used as the leukotriene inhibitor, however, the pharmacological effect of which is in inhibition of the leukotriene synthesis itself.

Further advantages arise if the leukotriene receptor antagonist includes montelukast and/or zafirlukast and/or pranlukast and/or the leukotriene synthesis antagonist includes zileuton. Montelukast, zafirlukast and pranlukast directly intervene in inflammatory processes by binding to the cysteinyl leukotriene receptors present in the airways with high affinity and selectivity and inhibiting the pro-inflammatory effect of leukotrienes by blocking them. Alternatively or additionally, the antineoplastic hydroxyurea derivative zileuton (Zyflo®) can be used as the leukotriene synthesis antagonist, which blocks the synthesis of leukotrienes from arachidonic acid by inhibition of the 5-lipoxygenase. However, it is to be emphasized that basically other than the mentioned leukotriene receptor antagonists or than the mentioned leukotriene synthesis antagonist can also be provided.

In a further advantageous development of the invention, it has proven to be advantageous if the pharmaceutical composition includes at least one further active agent from the group of the anti-inflammatory agents, antibiotics, antiasthmatics, bronchodilators, betasympathomimetic drugs, diuretics, parasympatholytics, vitamins, cytochrome P450 inductors and/or vaso-dilating agents in a pharmacologically effective amount. Hereby, in particular, the most frequent acute complications mentioned above under 1. to 4. can be specifically treated. The use of antibiotics for example allows the inhibition of bacterial infections. With the aid of diuretics, the risk of development of a pulmonary edema can advantageously be reduced. Therein, for example, it can be provided that the pharmaceutical composition concretely includes clarithromycin, pentoxifylline, vitamin A, provitamin A, hydrochlorothiazide, spironolactone, furosemide, salbutamol, ipratropium bromide, phenobarbital, phenytoin, rifampicin and/or at least one steroid, in particular a corticosteroid and/or a glucocorticoid, such as dexamethason, as a further active agent.

In further development of the invention, it has proven to be advantageous if the pharmaceutical composition is prepared in a dosage between (0.2±10%) mg/kg/d and (3.5±10%) mg/kg/d, in particular between (1.0±10%) mg/kg/d and (2.0±10%) mg/kg/d for administration of the at least one leukotriene inhibitor. Therein, in the entire present disclosure, the designation mg/kg/d signifies the amount of active substance to be administered in milligrams per kilogram of body weight of the patient and per day (d). Since prematurely born infants usually have a birth weight below 1500 g and partially of 500 g or less, a high effectiveness of the pharmaceutical composition with side effects as low as possible at the same time is assured by the mentioned dosage range. Therein, the preparation of the pharmaceutical composition can basically be selected depending on the desired form and frequency of administration.

By the pharmaceutical composition including a suitable amount of at least one pharmaceutically acceptable vehicle, improved dosability as well as simplified handling of the pharmaceutical composition can be achieved. The vehicle is preferably an inert vehicle such that undesired degradation of the pharmaceutically effective ingredients of the pharmaceutical composition is reliably excluded. Pharmaceutically acceptable carriers suitable for use in these compositions can for example include one or more binders, which are selected from a group including inert binders, wherein reactive binders such as lactose or other mono or disaccharides basically can also be provided.

A further aspect of the invention relates to a method for treating and/or for prophylaxis of a lung disease, in particular a bronchopulmonary dysplasia, in a prematurely born infant, in which a pharmacologically effective amount of at least one leukotriene inhibitor is administered to the prematurely born infant. Hereby, improved treatment for prematurely born infants is achieved, with the aid of which the progress of disease often crucial to life can be avoided or at least advantageously affected. The advantages of such a method can be taken from the previous explanations to the use according to the invention as well as to the pharmaceutical composition according to the invention. The preferred embodiments and developments presented in connection with the use according to the invention and the pharmaceutical composition according to the invention as well as the advantages thereof correspondingly apply to the method according to the invention and vice versa.

In an advantageous development of the invention, it is provided that at least one leukotriene receptor antagonist and/or at least one leukotriene synthesis antagonist is administered to the prematurely born infant as the leukotriene inhibitor. Leukotriene receptor antagonists bind to leukotriene receptors (LTD4 receptors) and thereby block the effect of the leukotrienes in the inflammation process. Hereby, an anti-inflammatory effect in the bronchial system of the prematurely born infant is achieved. Alternatively or additionally, a leukotriene synthesis antagonist can also be used as the leukotriene inhibitor, however, the pharmacological effect of which is in the inhibition of the leukotriene synthesis itself. Thus, the development of leukotrienes can advantageously be blocked via various mechanisms.

Further advantages arise if montelukast and/or zafirlukast and/or pranlukast are administered to the prematurely born infant as the leukotriene receptor antagonist and/or zileuton is administered to it as the leukotriene synthesis antagonist. Montelukast, zafirlukast and pranlukast directly intervene in inflammatory processes by binding to the cysteinyl leukotriene receptors present in the airways with high affinity and selectivity and inhibiting the pro-inflammatory effect of leukotrienes by blocking them. Alternatively or additionally, the antineoplastic hydroxyurea derivative zileuton (Zyflo®) can be used as the leukotriene synthesis antagonist, which blocks the synthesis of leukotrienes from arachidonic acid by inhibition of the 5-lipoxygenase. However, it is to be emphasized that other than the mentioned leukotriene receptor antagonists or than the mentioned leukotriene synthesis antagonist can basically also be provided.

Further advantages arise by supplying an increased energy dose to the prematurely born infant and/or by administering at least one further active agent from the group of the anti-inflammatory agents, antibiotics, antiasthmatics, bronchodilators, betasympathomimetic drugs, diuretics, parasympatholytics, vitamins, cytochrome P450 inductors and/or vaso-dilating agents to it. Due to the increased energy demand of the diseased prematurely born infant, increased energy dose by high-calorie nutrition has proven to be advantageous. Alternatively or additionally, with the aid of the further active agent, in particular the most frequent acute complications mentioned above under 1. to 4. can be specifically treated. The use of antibiotics for example allows the inhibition of bacterial infections. With the aid of diuretics, the risk of development of a pulmonary edema can advantageously be reduced.

Particular advantages arise if clarithromycin, pentoxifylline, vitamin A, provitamin A, hydrochlorothiazide, spironolactone, furosemide, salbutamol, ipratropium bromide, phenobarbital, phenytoin, rifampicin and/or at least one steroid, in particular a corticosteroid and/or a glucocorticoid such as dexamethason, is administered to the prematurely born infant as a further active agent. For example, with the aid of steroids such as dexamethason, a fast improvement of the pulmonary function and reduction of the occurrence of BPD can be achieved, wherein in contrast to the known therapy approaches, due to the synergistic effect with the leukotriene inhibitor, substantially lower dosages are necessary to achieve comparable effects. Accordingly, however, the frequency of side effects is advantageously reduced.

In a further advantageous development of the invention, it is provided that an administered dose of at least the leukotriene inhibitor is increased with respect to a dose upon administration without cytochrome P450 inductor, if a cytochrome P450 inductor is provided as a further active agent. Since leukotriene inhibitors—for example montelukast—are metabolized via the cytochrome P450 system (CYP3A4), active agents inducing CYP3A4 (e.g. phenobarbital, phenytoin or rifampicin) can decrease the plasma concentration and thus the effect of montelukast. In order to neutralize this effect, it has proven to be advantageous to increase the dose of the leukotriene inhibitor at least such that the increased degradation rate is compensated for and after administration a plasma concentration results corresponding to that of administration without cytochrome P450 inductor. For this purpose, for example, a pharmaceutical composition including the leukotriene inhibitor can be prepared correspondingly with an increased concentration or amount of leukotriene inhibitor. Alternatively or additionally, the leukotriene inhibitor can be administered separately from the cytochrome P450 inductor in corresponding dose. Analogously, the dose of further active agents affected by the increased degradation can of course also be correspondingly increased.

A particularly flexible prophylaxis and/or treatment method is allowed in further development of the invention in that the at least one leukotriene inhibitor and the at least one further active agent are administered collectively and/or temporally separately from each other.

Therein, in further development, it can be provided that at least the at least one leukotriene inhibitor is intermittently administered to the prematurely born infant is once, twice and/or several times per day over a suitable period of time and/or in which at least the at least one leukotriene inhibitor is continuously administered to the prematurely born infant over a suitable period of time. For example, the at least one leukotriene inhibitor can be administered once, twice, three, four, five, six times or more frequently in daily individual dosages or continuously, for example via a permanent drip infusion. In this manner, the plasma concentration or the temporal progress of the plasma concentration can be specifically adapted to the respectively present disease pattern.

By orally and/or intravenously and/or aerogenously and/or rectally administering at least the at least one leukotriene inhibitor to the prematurely born infant, a particularly flexible possibility of administration of the pharmaceutically active agent is given.

In further development of the invention, it has proven to be advantageous if the at least one leukotriene inhibitor is administered to the prematurely born infant in a dosage between (0.2±10%) mg/kg/d and (3.5±10%) mg/kg/d, in particular between (1.0±10%) mg/kg/d and (2.0±10%) mg/kg/d. Since prematurely born infants usually have a birth weight below 1500 g and partially of 500 g or less, a high effectiveness of the pharmaceutical composition with side effects as low as possible at the same time is ensured by the mentioned dosage range. Preferably, a pharmaceutical composition correspondingly prepared depending on the desired administration form and frequency is administered to the prematurely born infant.

A further improvement of the prophylaxis and/or treatment method is allowed in further development in that the at least one leukotriene inhibitor is administered to the prematurely born infant in the first treatment week in a dose of (1.0±10%) mg/kg/d, in the second treatment week in a dose of (1.5±10%) mg/kg/d and in the third treatment week in a dose of (2.0±10%) mg/kg/d. Hereby, a particularly high compatibility and low level of side effects is ensured, since already with low dosages an advantageous therapeutic effect is ensured on the one hand, and upon occurrence of incompatibilities dependent on dose, the dosage increase is cancelled and the treatment or prophylaxis can be continued with lower dosage on the other hand.

In a further advantageous development of the invention, it is provided that the at least one leukotriene inhibitor is administered over a period of time of about eight weeks, if the prematurely born infant suffers from a moderate lung disease, or that the at least one leukotriene inhibitor is administered over a period of time of about twenty-four weeks if the prematurely born infant suffers from a severe lung disease. Therein, by a period of time of about eight weeks, between seven and nine weeks, by a period of time of about twenty-four weeks, between twenty-three and twenty-five weeks are to be understood, wherein moreover additional deviations of up to five days can be provided. As the clinical symptoms for the severity of the disease, for example, increased breathing frequency, intensified stressed breathing with retractions on the thorax, increased bronchial secrete, cough, growth delay and livid skin and mucosa can be taken. Severe lung diseases can be identified in that diffuse overinflation zones are found besides insufficiently aerated zones (atelectases) in the x-ray image of the lung, among other things. Diagnosis and classification of the severity of the lung disease can also be effected based on the oxygen demand required for sufficient oxygen saturation of the blood at a specified age of the infant. According to definition, therein, the oxygen demand at the time of a corrected age of 36 weeks of pregnancy (SSW) is decisive. One differentiates between a mild lung disease (with 36 SSW no longer increased oxygen demand), a moderate lung disease (less than 30% oxygen required in the inhaled air) and a severe lung disease (more than 30% and/or ventilation or breathing support required). The severity of the lung disease is usually determined based on the so-called NICHD score (mild, moderate, severe BPD). For the phase of decision of which severity is present, a period of time of up to four weeks can be provided per patient. If it becomes apparent that the prematurely born infant is only subjected to mild BPD, it can be provided that it is desisted from administration of at least the leukotriene inhibitor.

Further features of the invention are apparent from the claims and the embodiment. The features and feature combinations mentioned above in the description as well as the features and feature combinations mentioned below in the embodiment are usable not only in the respectively specified combination, but also in other combinations or alone without departing from the scope of the invention. In the description of the embodiment, there are used as abbreviations:

BPD: bronchopulmonary dysplasia

CPAP: continuous positive airway pressure

d: day

FiO₂: inspiratory oxygen fraction

FG: prematurely born infant

kg: kilogram

mg: milligram

NICHD: National Institute of Child Health and Human Development

PMA: postmenstrual age

SSW: week of pregnancy

Within the scope of the presently described healing attempt, within the scope of a standardized project, data was acquired in several neonatal departments, from which it is apparent that by the treatment with a leukotriene inhibitor compared to previous forms of therapy, a less invasive anti-inflammatory therapy with less side effects can be achieved in these patients.

In the first phase, the severity of the lung disease of several prematurely born infants was determined. The lung disease was bronchopulmonary dysplasia, which was categorized based on the NICHD score (mild, moderate, severe BPD). For assessing the severity of the bronchopulmonary dysplasia, the pulmonary acuity score (PAS) can also be gathered among other things. The PAS represents a pulmonary score for assessing the severity of a bronchopulmonary dysplasia and is defined as the inspiratory oxygen fraction (FiO₂) multiplied by the support score, added with the medical score. The PAS includes the following parameters: FiO₂, mode of ventilation as well as conventional medication for treating a bronchopulmonary dysplasia. This score known per se ranges from 0.21 to 2.95. The smaller the PAS value, the better the pulmonary outcome is estimated.

For determining the severity, a period of time of four weeks was scheduled per patient. If it became apparent that the prematurely born infant is only subject to mild BPD, the pharmaceutical composition with the leukotriene inhibitor was not offered to this group of patients.

A leukotriene inhibitor was administered to patients with moderate BPD over eight weeks. The subsequently gathered clinical and laboratory chemical parameters could—as far as present—be used for comparative purposes. The pharmaceutical composition with the leukotriene inhibitor was delivered to patients with severe BPD and thus infaust prognosis over a longer period of time of six months. Therein, side effects could not be observed in any of the patients treated heretofore.

A randomization of this experiment was effected in the manner that the pharmaceutical composition with the leukotriene inhibitor was offered to all of the critically diseased patients and the group with absent approval of the legal guardians was used as a clinical control group. In all of the patients, the parameters usually routinely acquired in the neonatology were transferred to separate documentation sheets in regular intervals and subjected to evaluation. Subsequent to the 24 first weeks of therapy, a further post-observation phase lasting further 24 weeks was performed.

The pharmaceutical composition with the leukotriene inhibitor was administered according to the following dose regimen:

If undesired side effects did not occur, in each patient, starting from a start dose of about 1.0 mg/kg body weight/day (d), the dosage of the leukotriene inhibitor was increased by 0.5 mg/kg body weight/d to 1.5 mg/kg body weight/d after one week.

After a further week, the dose was then again increased by 0.5 mg/kg body weight/d to a maximum dose of about 2.0 mg/kg body weight/d. Montelukast, zafirlukast, pranlukast and zileuton were provided individually and in any combinations as the leukotriene inhibitors. Previously, a selection of those patients, which belonged to a high-risk collective for development of a bronchopulmonary dysplasia, was effected. Patients belong to this high-risk collective:
with a date of birth ≦32^{nd} week of pregnancy;
with a birth weights 1500 g;
with a weight below the 3^{rd} weight percentile (VLBW, very low birth weight) without initial presence of an RDS (respiratory distress syndrome);
with a progressive respiratory insufficiency from the 2^{nd} week of life;
which have the following anamnesis criterions: systemic infection, persistent ductus arteriosus, colonization with specific microorganisms (ureaplasma, CMV); and/or
which have a milky cloudiness of the lung parenchyma in the x-ray thorax (from the second week of life).

For the therapy with a leukotriene inhibitor, after screening of the above mentioned risk factors, only patients were considered:
which were born before/in the 32^{nd} week of pregnancy;
which still require FiO₂>0.21>28^{th} day of life; and
which require FiO₂>0.21≧36^{th} week postmenstrual age (PMA).

As exclusion criteria for the healing attempt, there were determined:
Blood coagulation disorder;
Presence of newborn seizures;
Presence of increased serum transaminases;
Presence of skin diseases; and
Therapy with phenobarbital.

Since phenobarbital is a cytochrome P450 inductor, alternatively, it could also be provided to prepare the pharmaceutical composition including the leukotriene inhibitor correspondingly with an increased concentration or amount of leukotriene inhibitor or to administer the leukotriene inhibitor in correspondingly increased dosage in order to compensate for the increased degradation rate.

Herein, as an accompanying therapy, it can basically be provided that all of the patients with bronchopulmonary dysplasia are additionally treated according to the usual therapy principles. The standard therapy of the BPD involves the following steps according to the severity of the BPD, which can be additionally performed individually or in any combination:
Prevention with prenatal, systemic glucocorticoid administration in the mother;
Oxygen application;
Liquid restriction;
Diuretics (hydrochlorothiazide, spironolactone and furosemide, respectively);
Increased energy supply;
Inhalation therapy (salbutamol and ipratropium bromide, respectively);
Systemic administration of glucocorticoids;
Avoidance of a barotrauma of the lung by ventilation as gentle as possible;
Early resorting to CPAP ventilation;
Generous indication for surfactant substitution;
Vitamin A administration;
Administration of bronchodilators; and
Antibiotics therapy.

Data was routinely acquired of all of the patients, which participate in the healing attempt, in the first eight weeks after beginning of the therapy. In the further course, the documentation—as far as possible—extended to all four weeks. The specified acquisition periods of time were in a time window of ±3 days in the two-week examinations, in the monthly controls, the acquisition period of time was at ±7 days.

The following parameters were acquired in the rounds of the slight BPD:

1. Physical examination and body weight, body height, blood pressure, heart rate, general condition;

2. Peripheral oxygen content of the blood (SpO₂); and

3. Period of hospitalization in days.

The following parameters were acquired in the rounds of the moderate and severe BPD:

1. Physical examination and body weight, blood pressure (systolic/diastolic and medium pressure), heart rate, general condition;

2. Peripheral oxygen content of the blood (SpO₂);

3. FiO₂ (facultative);

4. Arterial/capillary blood gas analysis (pH, pCO₂, pO₂, BE) (facultative);

5. Mode of ventilation (facultative);

6. Ventilation pressure (peak pressure, medium pressure) (facultative);

7. PEEP (facultative);

8. Ventilation duration in days;

9. X-ray thorax (facultative);

10. Period of hospitalization in days; and

11. Death (time of death, age in days of life).

It has to be noticed that the above mentioned parameters were acquired exclusively from already present examinations, but never additionally, i.e. outside of routine determinations.

The final evaluation was effected both in the moderate BPD (FiO₂<0.30 with 36 weeks PMA or upon discharge to home) and in the severe BPD (FiO₂>0.30 and/or ventilation or CPAP with 36 weeks PMA or upon discharge to home) a half year after completion of the therapy phase, respectively. The two treatment groups as well as the respective group without additional therapy with a leukotriene inhibitor such as for example montelukast were compared with respect to the following parameters (if present):

1. Period of hospitalization (d);

2. Ventilation duration (d);

3. Mode of ventilation;

4. Ventilation pressure (peak/medium pressure) (mmHg);

5. PEEP (mmHg);

6. Peripheral oxygen content of the blood (SpO₂);

7. Arterial/capillary pCO₂ (mmHg);

8. X-ray thorax (stages I-IV according to Northway);

9. Body weight (kg), blood pressure (mmHg), heart rate (/min);

10. Death (time of death, age in days of life);

11. PAS value; and

12. Occurrence/type of undesired events.

Basically, these parameters should at least remain the same or be superior to the corresponding parameters upon application of the previous mode of therapy. Evaluation was effected based on clinical aspects as well as based on the calculation and the comparison of the PAS value. With the aid of non-parametric test methods (Wilcoxon test for matching pairs), the PAS values of the weeks 2, 4, 6, 8, 12, 16, 20 and 24 were compared to week 0 as well as to each other and checked for significance.

In four such patients in weight between 460 g and 890 g, the healing attempt was performed with a pharmaceutical composition including montelukast as the leukotriene inhibitor. Therein, montelukast was administered within the scope of the healing attempt in a dose of 1-2 mg/kg body weight/d in the above described manner over 3-12 weeks. For this purpose, the pharmaceutical composition was prepared as mini tablets, since with a weight between 0.45 kg and 0.89 kg, a daily dosage of 2×0.25 mg up to 2×0.45 mg was required. In none of the treated patients, side effects occurred. In all of the infants, the pulmonary function improved. However, two infants died of the disease later after discontinuation of the medicament, two patients were completely healed. However, in a collective with expected lethality of 100%, this represents a considerable and surprising treatment success.

The parameter values specified in the documents for definition of process and measurement conditions for the characterization of specific characteristics of the object of the invention, are to be considered as encompassed by the scope of the invention, even within the scope of deviations—for example due to measurement errors, system errors, weighing errors, DIN tolerances and the like.

1. A method for preparing a pharmaceutical composition for prophylaxis and/or treatment of lung diseases in prematurely born infants, the method comprising combining at least one leukotriene inhibitor with at least one pharmaceutically acceptable vehicle.

2. The method according to claim 1, wherein the leukotriene inhibitor includes at least one leukotriene receptor antagonist and/or at least one leukotriene synthesis antagonist.

3. The method according to claim 2, wherein at least one of montelukast, zafirlukast, and pranlukast is used as the leukotriene receptor antagonist and/or zileuton is used as the leukotriene synthesis antagonist.

4. Use The method according to claim 1, wherein the pharmaceutical composition further comprises at least one further active agent selected from the group consisting of anti-inflammatory agents, antibiotics, antiasthmatics, bronchodilators, betasympathomimetic drugs, diuretics, parasympatholytics, vitamins, cytochrome P450 inductors, and vaso-dilating agents.

5. The method according to claim 4, wherein the further active agent is selected from the group consisting of clarithromycin, pentoxifylline, vitamin A, provitamin A, hydrochlorothiazide, spironolactone, furosemide, salbutamol, ipratropium bromide, phenobarbital, phenytoin, rifampicin steroid and combinations thereof.

6. The method according to claim 1, in which the pharmaceutical composition is prepared for oral, intravenous, aerogenous, or rectal administration.

7. A pharmaceutical composition for prophylaxis and/or treatment of lung diseases in prematurely born infants, the pharmaceutical composition comprising at least one leukotriene inhibitor.

8. The pharmaceutical composition according to claim 7, wherein the at least one leukotriene inhibitor includes a leukotriene receptor antagonist and/or a leukotriene synthesis antagonist.

9. The pharmaceutical composition according to claim 8, wherein the leukotriene receptor antagonist comprises at least one of montelukast zafirlukast, and pranlukast and/or the leukotriene synthesis antagonist comprises zileuton.

10. The pharmaceutical composition according to claim 7, the composition further comprising a pharmacologically effective amount of at least one further active agent selected from the group consisting of anti-inflammatory agents, antibiotics, antiasthmatics, bronchodilators, betasympathomimetic drugs, diuretics, parasympatholytics, vitamins, cytochrome P450 inductors, and vaso-dilating agents.

11. The pharmaceutical composition according to claim 7, wherein the composition is prepared in a dosage between (0.2±10%) mg/kg/d and (3.5±10%) mg/kg/d for administration of the at least one leukotriene inhibitor.

12. The pharmaceutical composition according to claim 7, the composition further comprising a suitable amount of at least one pharmaceutically acceptable vehicle.

13. A method for treating and/or for prophylaxis of a lung disease in a prematurely born infant, the method comprising administering a pharmacologically effective amount of at least one leukotriene inhibitor to the prematurely born infant.

14. The method according to claim 13, wherein the leukotriene inhibitor comprises at least one leukotriene receptor antagonist and/or at least one leukotriene synthesis antagonist.

15. The method according to claim 14, wherein the leukotriene receptor antagonist comprises at least one of montelukast, zafirlukast, and pranlukast and/or zileuton is the leukotriene synthesis antagonist.

16. The method according to claim 13, wherein the prematurely born infant is supplied with an increased energy dose and/or at least one further active agent selected from the group consisting of anti-inflammatory agents, antibiotics, antiasthmatics, bronchodilators, betasympathomimetic drugs, diuretics, parasympatholytics, vitamins, cytochrome P450 inductors and vaso-dilating agents.

17. The method according to claim 16, wherein the further active agent is selected from the group consisting of clarithromycin, pentoxifylline, vitamin A, provitamin A, hydrochlorothiazide, spironolactone, furosemide, salbutamol, ipratropium bromide, phenobarbital, phenytoin, rifampicin, steroid, glucocorticoid and combinations thereof.

18. The method according to claim 16, wherein an administered dose of at least the leukotriene inhibitor is increased with respect to a dose in administration without cytochrome P450 inductor, if a cytochrome P450 inductor is provided as a further active agent.

19. The method according to claim 16, wherein the at least one leukotriene inhibitor and the at least one further active agent are administered collectively and/or temporally separately from each other.

20. The method according to claim 13, wherein at least the at least one leukotriene inhibitor is intermittently administered to the prematurely born infant once, twice and/or several times per day over a suitable period of time and/or in which at least the at least one leukotriene inhibitor is continuously administered to the prematurely born infant over a suitable period of time.

21. The method according to claim 13, wherein at least the at least one leukotriene inhibitor is orally, intravenously, aerogenously, and/or rectally administered to the prematurely born infant.

22. The method according to claim 13, wherein the at least one leukotriene inhibitor is administered to the prematurely born infant in a dosage between (0.2±10%) mg/kg/d and (3.5±10%) mg/kg/d.

23. The method according to claim 13, wherein the at least one leukotriene inhibitor is administered to the prematurely born infant:
in the first treatment week in a dose of (1.0±10%) mg/kg/d;
in the second treatment week in a dose of (1.5±10%) mg/kg/d; and
in the third treatment week in a dose of (2.0±10%) mg/kg/d.

24. The method according to claim 13, wherein:
the at least one leukotriene inhibitor is administered over a period of time of about eight weeks, if the prematurely born infant suffers from a moderate lung disease; or in which
the at least one leukotriene inhibitor is administered over a period of time of about twenty-four weeks if the prematurely born infant suffers from a severe lung disease.
